(19) United States
(12) Patent Application Publication
(10) Pub. No.: US 2006/0004338 A1
(43) Pub. Date: Jan. 5, 2006

(54) Auxiliary product for use with a tampon

(76) Inventors: Annette Torkildsen, Vinterbro (NO); Sylvia Moller, Hakadal (NO)

Correspondence Address: William B. Patterson;Moser Patterson & Sheridan, 3040 Post Oak Boulevard, Suite 1500, Houston, TX, 77056, US

(21) Appl. No.: 10/533,486

(22) PCT Filed: Oct. 24, 2003
(86) PCT No.: PCT/NO03/00353

§ 371 Date: Apr. 28, 2005

(30) Foreign Application Priority Data
Nov. 1, 2002 (NO) 2002 5254

(51) Int. Cl. A61F 13/20

(52) U.S. Cl. 604/385.02; 604/14

(57) ABSTRACT

The present invention relates to a packaging for a tampon (1), in which the packaging (2; 10, 11) is formed such that it is suited to function as a finger protection during use of the tampon (1), whereby the packaging (2; 10, 11) prevents contact between fingers and vaginal fluids. The present invention also relates to a method for use of such packaging (2; 10, 11).

The present invention relates to an auxiliary product for use with a tampon, more specifically a device and method for packaging, inserting, and after use, dispose of a tampon.

A number of different kinds of tampons are commercially available today. The basic configurations of tampons are primarily similar and thoroughly tested. Different methods to insert a tampon without staining ones fingers exist. One product, with the trademark Tampax, utilizes a telescopic insertion mechanism that contributes to insert the tampon into the vagina, without a need for the fingers to get into contact with the vaginal fluids in the vagina. The mechanism works satisfactorily, but has certain disadvantages. Firstly, it may be difficult to position the tampon in the vagina in such a way that it cannot be felt. The tampon must not only be placed sufficiently far into the vagina, such that it is positioned beyond the muscles around the opening of the vagina and thereby causes discomfort, but it must also be oriented correctly inside the vagina. The Tampax mechanism makes it hard to position the tampon correctly, and this is one of the reasons many users prefer to employ tampons that require that the fingers come in contact with the vaginal fluids. Another drawback with the Tampax mechanism is that the combination of the insertion mechanism and the tampon results in a relatively long unit that takes up more space and requires more packaging material.

GB 2 227 666 A discloses a solution for the abovementioned problem, in which a skirt that makes up a finger protection is arranged on the rear end of, or around, the tampon. A finger may be inserted into the skirt and the tampon, whereupon the tampon may be inserted into the vagina and be correctly oriented, the skirt protecting the finger against the skin around the vagina. When the tampon is positioned correctly, the finger is drawn out, the skirt hanging down, possibly with a tab sticking outside the vagina, the tab functioning as an extraction string after use. The skirt that hangs out of the vagina opening may irritate the skin around the vagina, in addition to functioning as a leakage path for vaginal discharge and menstrual fluids.

The object of the present invention is to provide a solution that is not encumbered with the abovementioned drawbacks. This object is reached by means of a device of the kind that is characterized by the features indicated in the characterizing clause of claim 1 and of a method characterized by the features indicated in the characterizing clause of claim 6. Further advantageous features are indicated in the dependent claims.

The following description comprises non-limiting examples of advantageous embodiments of the present invention, with reference to the attached drawings, in which:

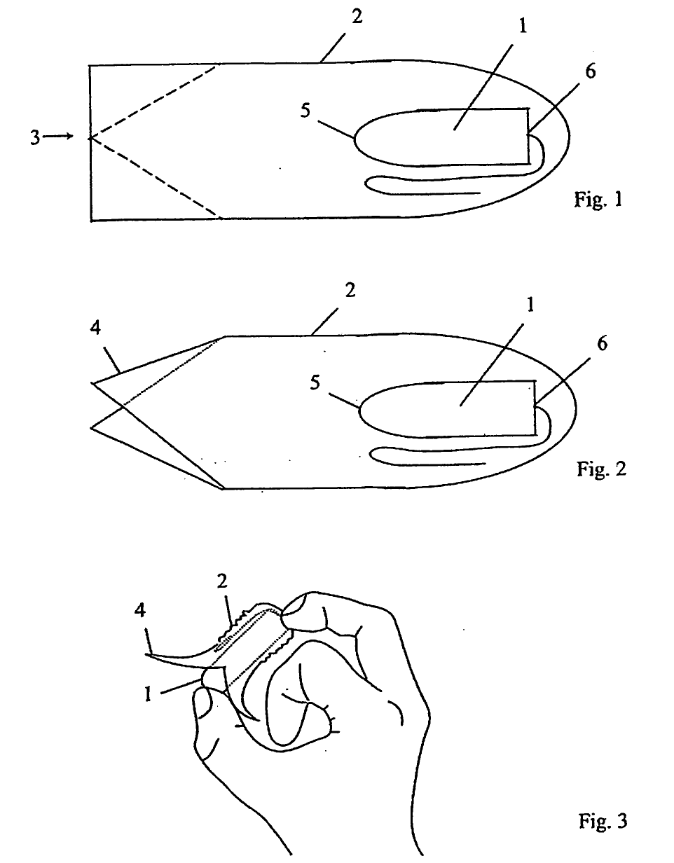

FIG. 1 shows a tampon that is sealed in one embodiment of a packaging according to the present invention;

FIG. 2 shows the tampon and packaging in FIG. 1, where the packaging is opened;

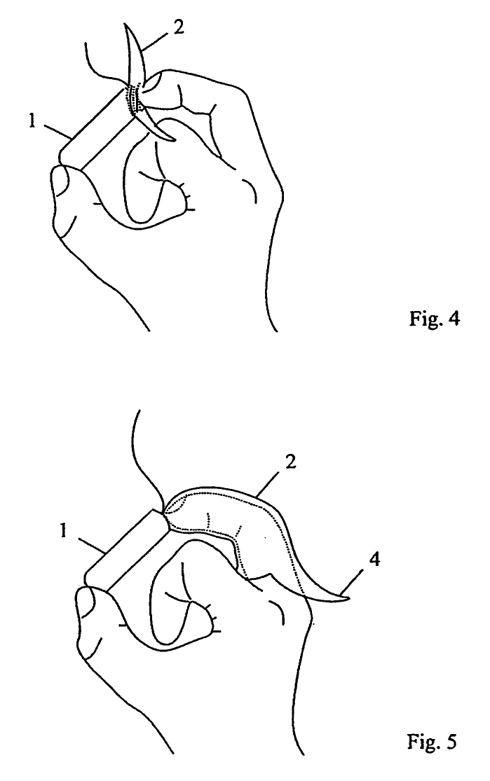

FIG. 3-5 shows how the packaging according to one embodiment of the invention may be used;

FIG. 6 shows a tampon that is sealed in another embodiment of the present invention, in which this embodiment comprises a two-part packaging; and

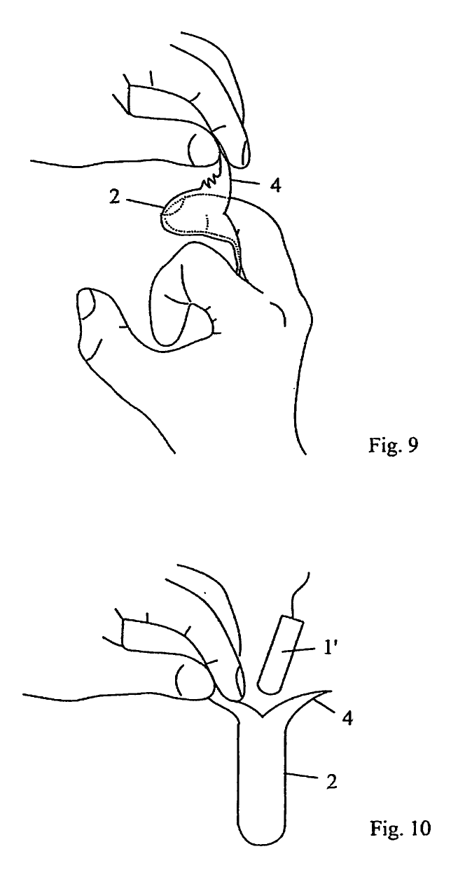

FIG. 7 shows the tampon and the packaging in FIG. 6, where the outer packaging is opened.

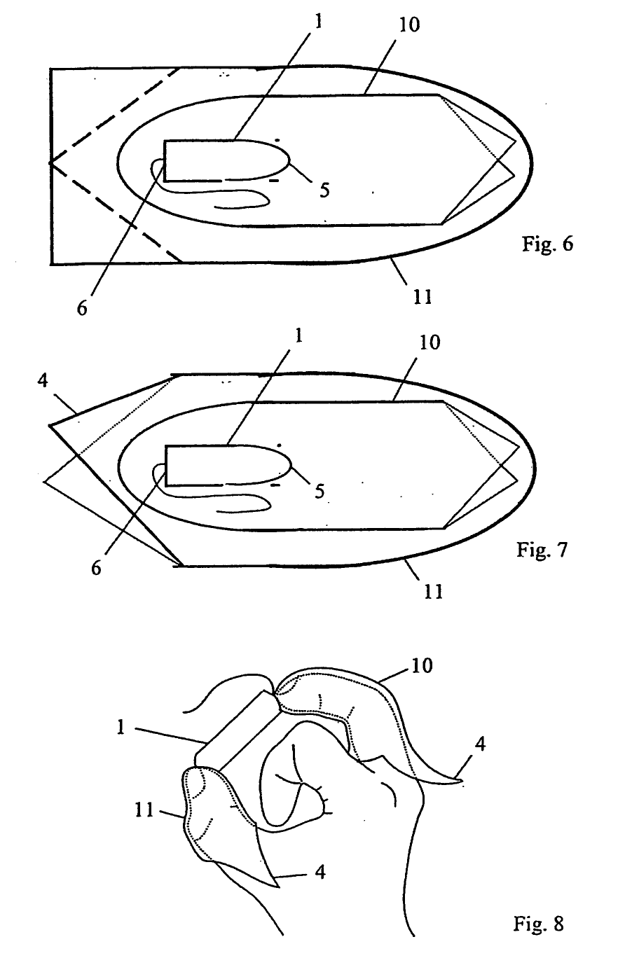

FIG. 1 shows a tampon 1 that is sealed in a packaging 2. In one embodiment of the packaging 2, an aperture 3 is arranged such that two tabs 4 are formed when the packaging has been opened (FIG. 2). These tabs 4 make it easier to handle the packaging during use. When the packaging has been opened, the tampon is grabbed as shown in FIG. 3, with the thumb on the tampon point 5 and the index finger or middle finger on the rear end 6 of the tampon. Then the packaging 2 is turned inside out over the index finger or possibly middle finger as shown in FIG. 4 and 5, the packaging 2 now forming a finger protection or glove. The tampon 1 may now be inserted into the vagina in a correct manner, without the fingers coming into contact with the vaginal fluids.

If a used tampon is replaced, the used tampon may be disposed of in the packaging 2 by carrying out the same steps outlined above, only in reverse.

The packaging will thus function both as sealing means, a finger protection and a disposal bag. This may be of great advantage, especially during situations with bad sanitary conditions, such as cabin trips, camping trips or in public places such as bars, restaurants, gyms etc.. In addition, the disposal bag function of the packaging contributes to reduce littering. Since the packaging functions as a disposal bag in itself, the total amount of litter is reduced to a minimum and it is not spread about.

FIGS. 6 to 8 show a second embodiment of the present invention, in which the tampon 1 is packed into two bags 10, 11. The outer bag or packaging 11 may thus be used as a finger protection for the index finger or middle finger, and the inner bag or packaging 10 forms a finger protection for the thumb, or vice versa. With this embodiment it may possibly be easier to handle a used tampon without getting into finger contact with menstrual fluids during disposal.

A very important advantage of the present invention is that the tampon may be handled in a completely sterile manner, because the tampon under no circumstance comes into contact with bare fingers that otherwise may contribute to transmittal of unwanted bacteria or contamination to the vaginal area.

During use of this second embodiment of the present invention, the same steps are generally followed as with the first embodiment shown above, except that the second embodiment comprises two bags or packages 10, 11, one outside the other, where only the outer bag or package 11 has to be sealed. The outer bag or package 11 is opened and pulled onto the index finger or middle finger, and the inner bag or packaging 10 is pulled onto the thumb, or vice versa. When the new tampon 1 is positioned correctly, the old tampon is grabbed by the to fingers that are covered by the bags or finger protection 10, 11, without any risk of getting into contact with vaginal fluids, whereupon the bags or finger protection are pulled over the used tampon, first one (10), then the other (11), until the used tampon finally is completely wrapped up and easily may be stuck into a pocket or a purse for later disposal in a suitable manner, for example into a trash can. In addition, the present invention does not result in loose and ripped up pieces of packaging, as with the currently available tampons.

It is understood that the packaging 10, 11 may be produced by any suitable material, primarily a plastic or rubber-like material that is strong and flexible enough to work in a reliable manner. The material must also be adapted to the sealing method that is chosen, this being welding, use of an adhesive etc., in addition to that it should be adapted for easy opening and possible forming of functional tabs 4. It may also be possible to use a locking mechanism of a “zip-lock” kind, enabling the packaging to be sealed after use. It is understood that the possible tabs 4 do not have to take on the shape that is shown in FIG. 2 and 7, but just as easily may be rounded, cornered etc., or the packages may only be split along parts of the edges.

The tampons used in conjunction with the present invention may be of any kind. The bags or packages 2, 10, 11 may be adapted to any kind and size of tampon.

In the claims:

1. Tampon and finger protecting packaging preventing contact between the fingers and menstrual fluids, the packaging comprising at least one sealed bag that keeps the tampon sterile before use, the packaging further being adapted to cover at least one finger, preventing the at least one finger from coming into contact with the tampon and thereby contaminating the tampon while inserting the tampon, or preventing the at least one finger from coming into contact with menstrual fluids that may contaminate the at least one finger,
characterized in that the packaging comprises at least one bag of a plastic material that does not damage while opening and after opening is adapted for use as a finger glove by turning the bag inside out over the at least on finger.

2. Tampon and finger protecting packaging according to claim 1,
characterized in that the packaging comprised two bags, one bag covering the other bag while packed:

3. Tampon and finger protecting packaging according to claim 1,
characterized in that the bag or bags comprise tabs, the tabs facilitating use of the packaging as finger protection.

4. Tampon and finger protecting packaging according to claim 1,
characterized in that the seal of the packaging is formed by welding, use of adherents, or a zip-lock type closing mechanism.
